(19) United States
(12) Patent Application Publication
(10) Pub. No.: US 2025/0285737 A1
(43) Pub. Date: Sep. 11, 2025

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Ryoichi ONO, Utsunomiya (JP); Masahiro YOSHIWAKI, Otawara (JP); Taro TAKOSHIMA, Nasushiobara (JP); Daigo OKUHATA, Sakura (JP); Hiroyuki YAMASAKI, Yaita (JP)

(73) Assignee: CANON MEDICAL SYSTEMS CORPORATION, Otawara-shi (JP)

(21) Appl. No.: 19/070,593

(22) Filed: Mar. 5, 2025

(30) Foreign Application Priority Data
Mar. 7, 2024 (JP) 2024-034929

(51) Int. Cl. G16H 30/20; G16H 30/40

(52) U.S. Cl. CPC G16H 30/20; G16H 30/40

(57) ABSTRACT

A medical information processing device of an embodiment includes processing circuitry. The processing circuitry automatically is configured to select, from among a plurality of applications, an execution application to be executed on a medical image that is a processing target, execute the execution application, set execution information regarding execution of processing of the processing target by the execution application on the basis of a result of referring to an examination end determination rule for supplementary information attached to the medical image, and update the examination end determination rule on the basis of a past result of processing by the execution application.

### CROSS-REFERENCE TO RELATED APPLICATION

The present application claims priority based on Japanese Patent Application No. 2024-034929 filed Mar. 7, 2024, the content of which is incorporated herein by reference.

### FIELD

Embodiments disclosed in this specification and drawings relate to a medical information processing device, a medical information processing method, and a storage medium.

### BACKGROUND

In the field of medical image diagnosis, analysis applications are provided that perform image processing on images captured by modalities such as X-ray CT devices and MRI devices to analyze the images. These analysis applications perform various analyses and computations on behalf of doctors and others. Medical institutions aim to improve the efficiency of medical treatment by introducing such analysis applications.

In addition, various analysis applications are provided according to the type of medical image and the purpose of processing (analysis processing), and a platform for selecting an analysis application that appropriately executes medical images captured by multiple modalities has been developed. For example, a modality outputs medical images to be used in an examination. In an examination, usually, multiple examination items (examination series, hereinafter referred to as series) are examined, and the modality outputs medical images for each series. The platform selects an analysis application that appropriately processes each of output medical images for each series.

For example, the first series may be a series that does not require analysis, the second series may be a series that can be appropriately processed by analysis application A, and the third series may be a series that can be appropriately processed by analysis application B. Or, all of the first through third series that are output may be series that can be appropriately processed by analysis application A.

In conventional platforms, for example, an analysis application is selected and processing is performed each time a series of medical images is processed. In this case, if all of the first through third series are series that can be appropriately processed by analysis application A, analysis application A will process the first through third series individually, which is inefficient.

To address this problem, for example, there are systems that perform control to start processing when a timeout occurs without a series that is processed by the same analysis application being output after one analysis application is selected. With this type of control, even if a series to be processed simultaneously is not output later, it is necessary to wait until processing by the analysis application times out, which is wasteful regarding the time required for overall processing and affects performance.

### BRIEF DESCRIPTION OF THE DRAWINGS

FIG. 1 is a diagram showing an example of a usage environment and functional blocks of a medical information processing device 100 according to an embodiment.

FIG. 2 is a diagram showing an example of a data structure of an examination end determination rule ERL.

FIG. 3 is a diagram showing an example of a DICOM tag output by a modality

FIG. 4 is a flowchart showing an example of a processing flow in the medical information processing device 100.

FIG. 5 is a flowchart showing an example of a processing flow in the medical information processing device 100.

FIG. 6 is a flowchart showing an example of a processing flow in the medical information processing device 100.

### DETAILED DESCRIPTION

Hereinafter, a medical information processing device, a medical information processing method, and a storage medium of an embodiment will be described with reference to the drawings.

The medical information processing device of an embodiment has processing circuitry. The processing circuitry is configured to automatically select an execution application to be executed on a medical image that is a processing target from among a plurality of applications, execute the execution application, set execution information regarding execution of processing of the processing target by the execution application on the basis of a result of referring to an examination end determination rule for supplementary information attached to the medical image, and update the examination end determination rule on the basis of a past result of processing by the execution application.

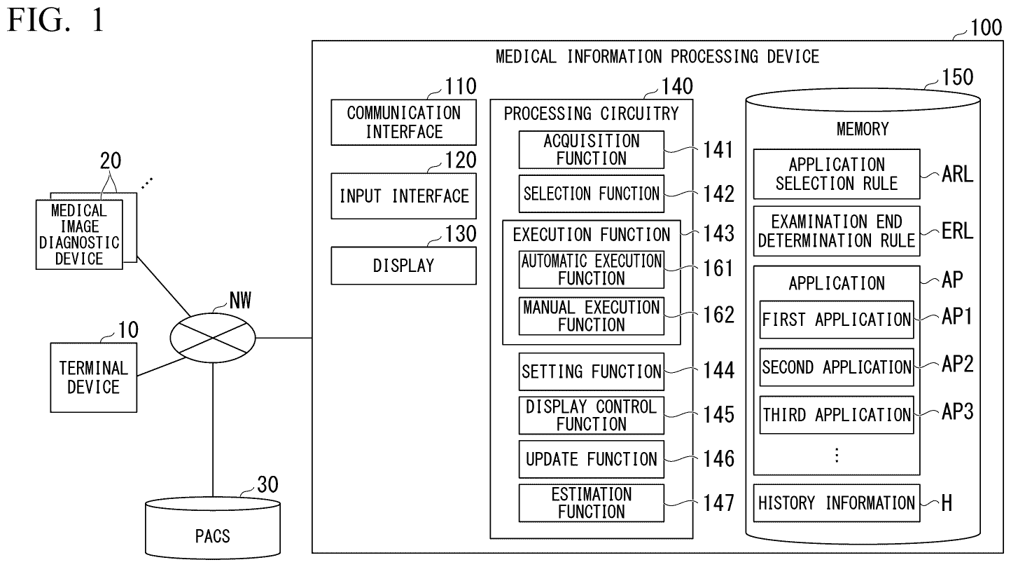

FIG. 1 is a diagram showing an example of a usage environment and functional blocks of a medical information processing device 100 according to an embodiment. The medical information processing device 100 provides a function of executing each of a plurality of analysis applications (hereinafter also referred to as applications) and a function of selecting an application that matches a series supplied by a modality.

Applications perform various types of analysis processing according to the purpose on a medical image to be analyzed. The applications include, for example, clinical applications for detecting lesions in patients, applications for performing site segmentation, applications for checking tumors, applications for determining the progression of lesions, applications for detecting locations of specific lesions such as cerebral infarction, and the like. The applications are, for example, machine learning models generated by learning processing using machine learning techniques such as deep learning.

The medical information processing device 100 is placed in, for example, a medical institution such as a hospital. The medical information processing device 100 may be, for example, a workstation, a server, or the like. The medical information processing device 100 is connected to, for example, at least one terminal device 10, at least one medical image diagnostic device (hereinafter, modality) 20, a picture archiving and communication system (PACS) 30, and the like via a communication network NW such that data can be transmitted and received therebetween.

The communication network NW refers to a general information and communication network that uses electrical communication technology. The communication network NW includes telephone communication line networks, optical fiber communication networks, cable communication networks, and satellite communication networks in addition to wireless/wired local area networks (LANs) such as a hospital backbone LAN and Internet networks.

The terminal device 10 is a device for using functions of an AI platform provided by the medical information processing device 100. The terminal device 10 is, for example, a personal computer, or a mobile terminal such as a tablet or a smartphone. The terminal device 10 is operated, for example, by a doctor, an engineer, or the like. A dedicated application program or a browser is started on the terminal device 10, and various types of information provided by the medical information processing device 100 is provided to the doctor, or the like.

The modality 20 captures an image of a patient (subject) to be diagnosed and generates a medical image. The modality 20 is, for example, an X-ray computed tomography (CT) device, an X-ray diagnostic device, a magnetic resonance imaging device, an ultrasound diagnostic device, a nuclear medicine diagnostic device, or the like. The modality 20 generates an image to which supplementary information is added.

The modality 20 generates, for example, images that comply with the Digital Imaging and Communication in Medicine (DICOM) standard (hereinafter, DICOM images). A DICOM tag is added to DICOM images as supplementary information. The DICOM tag includes examination information related to an examination. The examination information includes, for example, the processing priority, imaging time, device ID for identifying the modality, examination site, presence or absence of a contrast agent, imaging protocol number, a protocol name, patient ID for identifying the patient, examination ID for identifying the examination, information on the facility where the device is placed, device manufacturer, and information (“series description”) manually input by a device operator (engineer or the like), etc.

The PACS 30 stores various images generated by the modality 20. The PACS 30 stores, for example, CT images, magnetic resonance (MR) images, ultrasound images, and the like for each patient. The PACS 30 is realized, for example, by a semiconductor memory element such as a random access memory (RAM) or a flash memory, a hard disk, or an optical disc.

The medical information processing device 100 includes, for example, a communication interface 110, an input interface 120, a display 130, processing circuitry 140, and a memory 150. The communication interface 110 communicates with external devices such as the terminal device 10, the modality 20, and the PACS 30 via a communication network NW. The communication interface 110 includes, for example, a communication interface such as a network interface card (NIC).

The input interface 120 accepts various input operations from the operator of the medical information processing device 100, converts the accepted input operations into electrical signals, and outputs the electrical signals to the processing circuitry 140. For example, the input interface 120 includes a mouse, a keyboard, a trackball, a switch, a button, a joystick, a touch panel, etc. The input interface 120 may be, for example, a user interface that accepts voice input, such as a microphone.

In this specification, the input interface is not limited to an input interface having physical operating parts such as a mouse and a keyboard. For example, examples of the input interface may also include electrical signal processing circuits that receive an electrical signal corresponding to an input operation from an external input apparatus provided separately from the device and output the electrical signal to a control circuit.

The display 130 displays various types of information. For example, the display 130 displays an image generated by the processing circuitry 140, a graphical user interface (GUI) for receiving various input operations from an operator, and the like. For example, the display 130 is a liquid crystal display (LCD), a cathode ray tube (CRT) display, an organic electroluminescence (EL) display, or the like.

The processing circuitry 140 includes, for example, an acquisition function 141, a selection function 142, an execution function 143, a setting function 144, a display control function 145, an update function 146, and an estimation function 147. The processing circuitry 140 realizes these functions, for example, by a hardware processor (computer) executing a program stored in the memory 150 (storage circuit).

The hardware processor refers to circuitry such as a central processing unit (CPU), a graphics processing unit (GPU), an application specific integrated circuit (ASIC), a programmable logic device (e.g., a simple programmable logic device (SPLD) or a complex programmable logic device (CPLD), or a field programmable gate array (FPGA)). Instead of storing the program in the memory 150, the program may be directly built into the circuitry of the hardware processor. In this case, the hardware processor realizes the functions by reading and executing the program built into the circuitry. The aforementioned program may be stored in advance in the memory 150, or may be stored in a non-transitory storage medium such as a DVD or CD-ROM, and may be installed in the memory 150 from the non-transitory storage medium by setting the non-transitory storage medium in a drive device (not shown) of the medical information processing device 100. The hardware processor is not limited to being configured as a single circuit, and may be configured as a single hardware processor by combining a plurality of independent circuits to realize each function. Further, a plurality of components may be integrated into a single hardware processor to realize each function.

The acquisition function 141 acquires DICOM data transmitted by the modality 20 or the PACS 30 via the communication network NW. The DICOM data is data including a DICOM image. The acquisition function 141 stores the acquired DICOM data in the memory 150.

The selection function 142 automatically selects an application (hereinafter, an execution application) to be executed on a DICOM image to be processed from among a plurality of applications in response to a request from the terminal device 10. The selection function 142 selects the execution application according to an application selection rule ARL, which is a rule engine stored in the memory 150. The selection function 142 is an example of a selection unit.

The execution function 143 includes, for example, an automatic execution function 161 and a manual execution function 162. The automatic execution function 161 executes the execution application selected by the selection function 142 on the DICOM image. The manual execution function 162 receives an instruction (hereinafter, manual execution instruction) to use the manual execution function 162 by a doctor or the like (hereinafter, user), which is transmitted by the terminal device 10. After receiving the manual execution instruction from the user, the manual execution function 162 executes the execution application on the DICOM image in response to an input operation by the user to the terminal device 10. The execution function 143 is an example of an execution unit.

The setting function 144 refers to the examination end determination rule for the DICOM tag attached to the DICOM image. The setting function 144 sets execution information regarding execution of processing of a processing target by the execution application on the basis of the result of referring to the examination end determination rule for the DICOM tag. The execution information includes, for example, a timing at which processing by the execution application starts (hereinafter, processing start timing), a timing at which processing by the execution application ends (hereinafter, processing end timing), and a timeout time when the acceptance time for accepting medical images to be processed collectively by the execution application ends. The setting function 144 is an example of a setting unit.

The display control function 145 transmits, to the terminal device 10, the processing result of the application executed by the execution function 143, the execution information set by the setting function 144, and the like. The display control function 145 transmits the execution information to the terminal device 10, thereby displaying the execution information on a display of the terminal device 10.

The update function 146 performs update processing for updating the examination end determination rule ERL on the basis of the result of processing by a past executed application. After the user issues a manual execution instruction, the update function 146 updates the examination end determination rule on the basis of the result of processing by the execution application that has been manually operated by the user. Alternatively, the update function 146 updates the examination end determination rule on the basis of the result of processing by the execution application after a certain period of time has elapsed since processing by the execution application ended. The update function 146 is an example of an update unit.

The estimation function 147 estimates a processing time required to process a DICOM image by the execution application. The estimation function 147 estimates the processing time on the basis of, for example, information on the DICOM image, such as the resolution and number of pixels of the DICOM image, information on the execution application, such as the content of processing in the execution application, and the like. The estimation function 147 is an example of an estimation unit.

The memory 150 is realized by, for example, a semiconductor memory element such as a RAM or a flash memory, a hard disk, or an optical disc. These non-transitory storage media may be realized by other storage devices connected via a communication network NW, such as a network attached storage (NAS) or an external storage server device.

The memory 150 may also include non-transitory storage media such as a read only memory (ROM) or a register. The memory 150 stores, for example, an application selection rule ARL, the examination end determination rule ERL, an application AP, history information H, and the like. The application AP includes a first application AP1, a second application AP2, a third application AP3, and the like, which perform various types of analysis processing depending on the purpose. Additionally, the memory 150 stores programs, parameter data, and other data used by the processing circuitry 140.

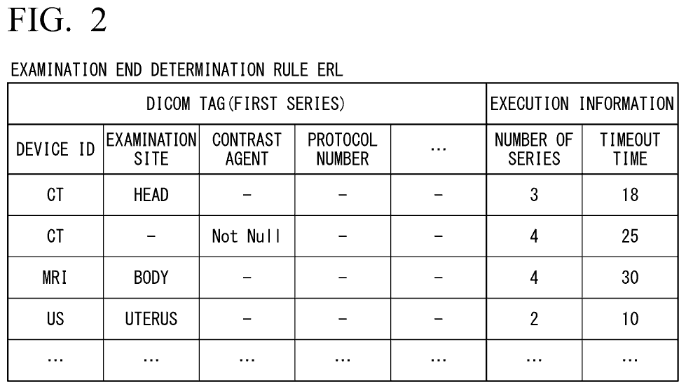

FIG. 2 is a diagram showing an example of a data structure of the examination end determination rule ERL. In the examination end determination rule ERL, reference information corresponding to the DICOM tag attached to an DICOM image output by the modality 20 is defined. The DICOM tag referred to in the examination end determination rule ERL is a DICOM tag attached to a DICOM image of a first series in examination series.

For example, the number of series in an examination and a timeout time are defined as the reference information. For example, when the device ID of the DICOM tag is “CT” and the examination site is “HEAD,” the number of series as execution information is defined as “3” and the timeout time is defined as 18 minutes. The reference information is read by the setting function 144 and used to set the execution information. The reference information is updated by the update function 146.

The setting function 144 may set the execution information on the basis of similarity between all of DICOM tags attached to the DICOM image output from the modality 20 and all of DICOM tags defined in the examination end determination rule ERL. For example, in the setting of the execution information, the setting function 144 uses a reference information associated with DICOM tags (a set of DICOM tags) in the examination end determination rule ERL most similar to DICOM tags attached to the DICOM image output from the modality 20. Known similarity determination techniques based on natural language processing or the like may be used. The setting function 144 may weight each of a plurality of DICOM tags (each of types), and calculate the similarity on the basis of the weighted plurality of DICOM tags. For example, the setting function 144 may weight each of a plurality of DICOM tags so that a contribution degree of a DICOM tag which identifies the content of examination itself (for example, a protocol name, series description, device ID, or the like) is high and a contribution degree of other DICOM tag (for example, patient ID, or the like) is low.

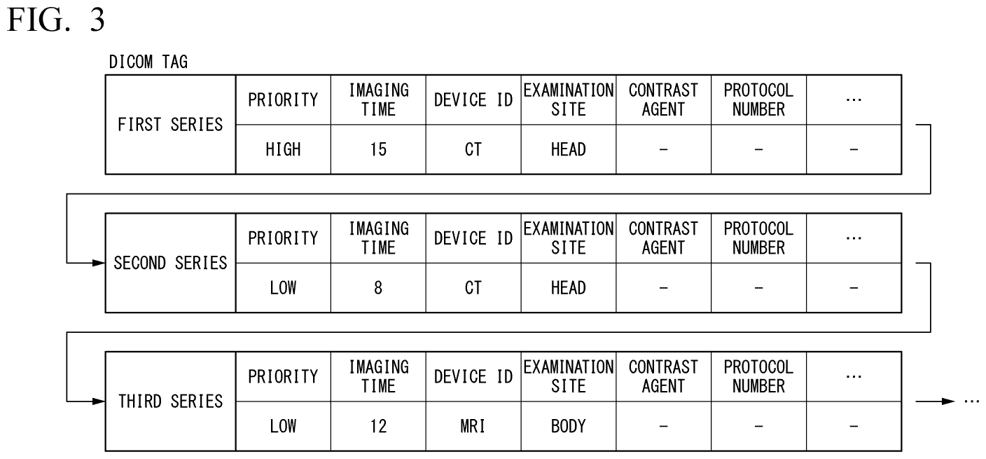

FIG. 3 is a diagram showing an example of a DICOM tag output by the modality 20. The DICOM tag is output each time a series is completed. The example shown in FIG. 3 shows DICOM tags up to the third series. Among the DICOM tags, “device ID,” “examination site,” “(presence or absence of) contrast agent,” and the like of the first series are referred to in the examination end determination rule ERL. The priority and imaging time are used to set execution information in the setting function 144.

After the user of the terminal device 10 issues a manual execution instruction, the history information H records the results of processing by the execution application manually operated by the user and the history of the examination end determination rule updated on the basis of the results of the processing. The history information H includes a plurality of results of processing and execution histories of the updated examination end determination rule.

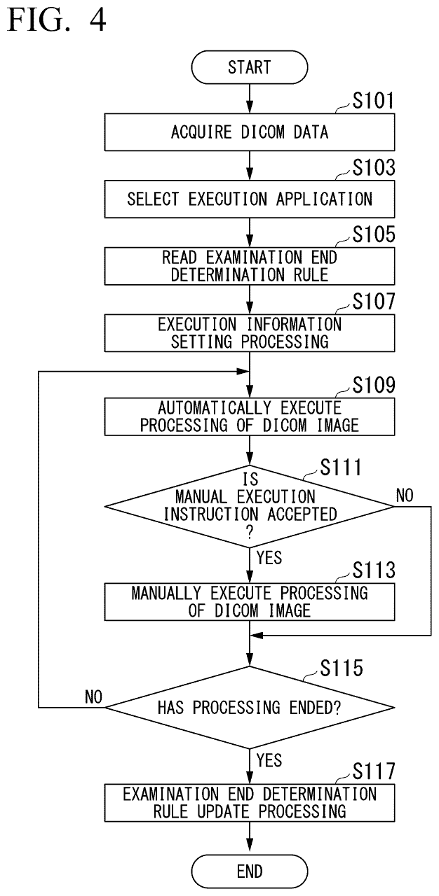

Next, various types of processing of the medical information processing device 100 according to the embodiment will be described. FIG. 4 is a flowchart showing an example of a processing flow in the medical information processing device 100. For example, the processing shown in FIG. 4 is executed when the medical information processing device 100 receives a processing request transmitted from the terminal device 10 on the basis of an operation of a doctor or the like.

First, the acquisition function 141 acquires DICOM data including a DICOM image and a DICOM tag of a first series of a patient to be analyzed from the PACS 30 in response to a processing request from the terminal device 10 (step S101) and stores the data in the memory 150. The acquisition function 141 acquires the DICOM data from the PACS 30 on the basis of, for example, a patient ID included in the processing request. When the terminal device 10 is used for emergency surgery or the like and a DICOM image captured by the modality 20 is displayed on the terminal device 10 as is, the acquisition function 141 acquires the DICOM data from the modality 20.

Then, the selection function 142 automatically selects an application suitable for processing the DICOM image as an execution application (step S103). The selection function 142 reads, for example, the application selection rule ARL stored in the memory 150. The selection function 142 compares the DICOM tag included in the acquired DICOM data with the read application selection rule ARL. The selection function 142 automatically selects an application suitable for processing the DICOM image as an execution application on the basis of the result of comparison between the DICOM tag and the application selection rule ARL.

Then, the setting function 144 reads the DICOM data and the examination end determination rule ERL stored in the memory 150 (step S105). The setting function 144 refers to the examination end determination rule ERL read from the memory 150 for the DICOM tag included in the DICOM data acquired by the acquisition function 141. The setting function 144 performs execution information setting processing for setting execution information at the time of executing the execution application on the basis of the result of referring to the examination end determination rule ERL for the reference DICOM tag (step S107).

In the execution information setting processing, the setting function 144 sets, for example, the number of examination series, the timeout time, the processing start timing, and the processing end timing. In the execution information setting processing, the setting function 144 may set some of the number of examination series, the timeout time, the processing start timing, and the processing end timing. The execution information setting processing will be described in further detail later.

Subsequently, the automatic execution function 161 automatically executes processing of the DICOM image in accordance with the execution information set by the setting function 144 (step S109). While the processing by the automatic execution function 161 is being executed, the manual execution function 162 determines whether or not a manual execution instruction transmitted by the terminal device 10 has been accepted (step S111).

If it is determined that a manual execution instruction has been accepted, the manual execution function 162 performs manual execution to execute the execution application on the DICOM image in response to an input operation by the user to the terminal device 10 (step S113). If it is determined that the manual execution function 162 has not accepted the manual execution instruction, the automatic execution function 161 determines whether automatic execution of DICOM image processing has ended (step S115).

If it is determined that automatic execution of DICOM image processing has not ended, the automatic execution function 161 returns the processing to step S109 and continues automatic execution. If the automatic execution function 161 determines that automatic execution of DICOM image processing has ended, the update function 146 performs update processing to update the examination end determination rule ERL (step S117). The update processing of the update function 146 will be described further later. In this manner, the medical information processing device 100 ends the processing shown in FIG. 4.

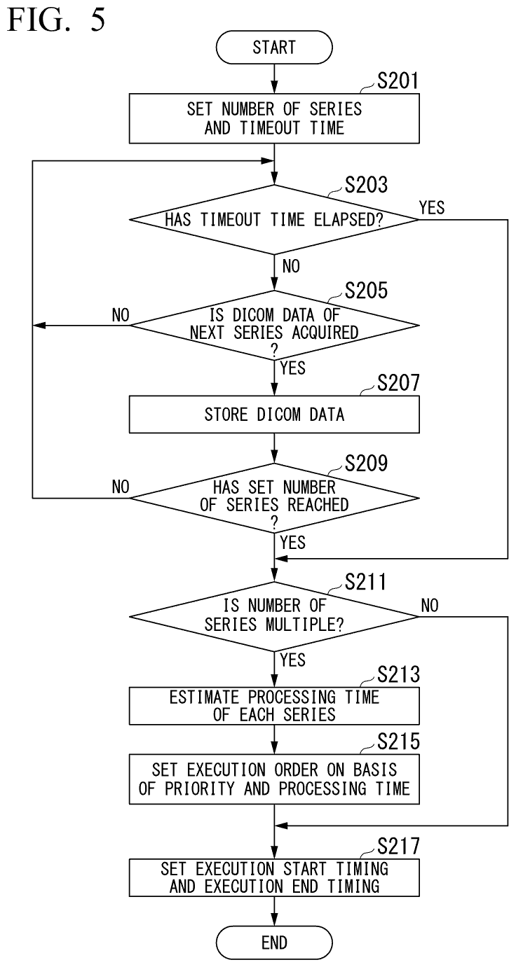

Next, the execution information setting processing will be described. FIG. 5 is a flowchart showing an example of a processing flow in the medical information processing device 100. In FIG. 5, an example of performing the execution information setting processing executed in step S107 of the flowchart in FIG. 4 in the medical information processing device 100 will be mainly described.

In the execution information setting processing, the setting function 144 refers to the examination end determination rule ERL for the DICOM tag acquired by the acquisition function 141 and sets the number of examination series and the timeout time (step S201). Subsequently, the setting function 144 determines whether the set timeout time has elapsed (step S203).

If it is determined that the set timeout time has not elapsed, the setting function 144 determines whether DICOM data of the next series has been acquired by the acquisition function 141 (step S205). If it is determined that DICOM data of the next series has not been acquired by the acquisition function 141, the setting function 144 returns the processing to step S203.

If it is determined that DICOM data of the next series has been acquired by the acquisition function 141, the acquisition function 141 stores the acquired DICOM data in the memory 150 (step S207). Subsequently, the setting function 144 determines whether the number of series for which DICOM data has been acquired has reached the number of series set in step S201 (step S209).

If it is determined that the number of series for which DICOM data has been acquired has not reached the set number of series, the setting function 144 returns the processing to step S203. If it is determined that the number of series for which DICOM data has been acquired has reached the number of series set in step S201, the setting function 144 proceeds with the processing to step S211. In addition, if it is determined in step S203 that the timeout time has been reached, the setting function 144 also proceeds with the processing to step S211.

The setting function 144 determines whether the number of examination series acquired by the acquisition function 141 is multiple (step S211). If the setting function 144 determines that the number of examination series acquired by the acquisition function 141 is not multiple (is single), the setting function 144 proceeds with the processing to step S217.

If the setting function 144 determines that the number of examination series acquired by the acquisition function 141 is multiple, the estimation function 147 estimates a processing time for each series on the basis of information on a DICOM image of each series and information on the execution application (step S213).

The setting function 144 then sets an execution order for each series on the basis of the priority included in the DICOM tag in the stored DICOM data and the processing time estimated by the estimation function 147 (step S215). The setting function 144 sets the execution order for each series, for example, in descending order of priority.

Alternatively, for example, when processing of a later acquired (hereinafter, subsequent) DICOM image by the execution application ends before the timing at which processing of an earlier acquired (hereinafter, preceding) DICOM image can be started (hereinafter, timing at which processing can start), the setting function 144 sets the timing of processing of the later acquired DICOM image to be before processing of the preceding DICOM image, regardless of the priority. Both the preceding DICOM image and the subsequent DICOM image can be processed by the execution application. The preceding DICOM image is an example of a first medical image, and the subsequent DICOM image is an example of a second medical image.

For example, it is assumed that the priority included in the DICOM tag of the first series is “high” and the imaging time is “15 minutes,” and the priority included in the DICOM tag of the second series is “low” and the estimated processing time is “5 minutes.” In this case, if only the priorities are compared, processing of the first series will be executed first, but the processing time of the second series is shorter than the imaging time of the first series. In this case, since processing of the second series can be executed while waiting for completion of imaging of the first series, processing of the second series is executed before processing of the first series.

The timing at which processing can start is calculated on the basis of, for example, the imaging time (hereinafter referred to as a preceding imaging time) included in the DICOM tag attached to the preceding DICOM image. The timing at which processing can start may be, for example, the timing at which the preceding imaging time has elapsed from the present, or the timing at which the preceding imaging time has elapsed from the time when the modality 20 started imaging of the preceding DICOM image, or a timing appropriately corrected from such time.

Subsequently, the setting function 144 sets an execution start timing and an execution end timing of each series on the basis of the set execution order and the processing time estimated by the estimation function 147 (step S217). In this manner, the medical information processing device 100 ends the processing shown in FIG. 5 and proceeds to step S109 in FIG. 4.

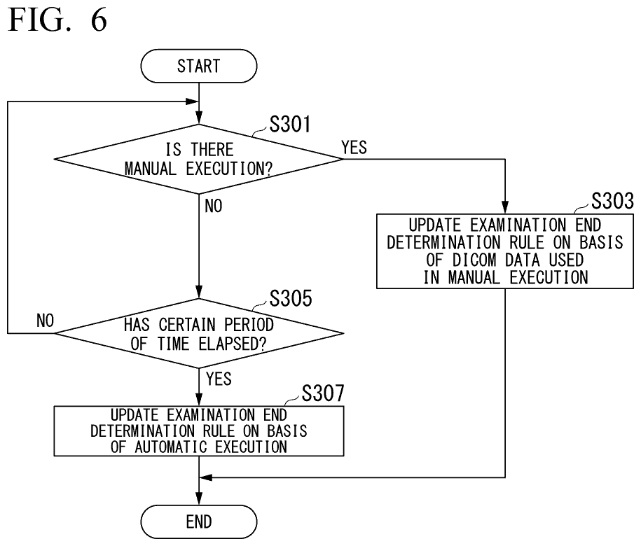

Subsequently, the examination end determination rule update process will be described. FIG. 6 is a flowchart showing an example of a processing flow in the medical information processing device 100. In FIG. 6, an example of the examination end determination rule update processing executed in step S117 of the flowchart in FIG. 4 in the medical information processing device 100 will be mainly described.

In the examination end determination rule update processing, the update function 146 first determines whether manual execution has been performed at the time of performing processing by the executed application (step S301). If manual execution has been performed, for example, the DICOM data (DICOM tag and DICOM image) of the first series used in the manual execution is analyzed, and the examination end determination rule ERL is updated on the basis of the analysis result (step S303).

If it is determined that there has been no manual execution at the time of processing by the execution application, the update function 146 determines whether a certain period of time has elapsed from the timing at which processing of the DICOM image of the series last acquired by the acquisition function 141 by the execution application has actually ended (hereinafter, the actual end timing) (step S305). The certain period of time may be set appropriately, for example, to a period of several hours such as one hour or five hours, one day, or several days.

If it is determined that the certain period of time has not elapsed from the actual end timing, the update function 146 returns the processing to step S301. If it is determined that the certain period of time has elapsed from the actual end timing, the update function 146 determines that the examination has ended, and, for example, analyzes the DICOM data of the first series used in the examination, and updates the examination end determination rule ERL on the basis of the analysis result (step S307).

For example, it is assumed that an examination includes two series, the first series includes a feature α, the second series does not include the feature α, and the execution application used for both the first and second series is application A. Here, it is assumed that history information H includes a history of executing processing of the first series by application A through automatic execution, and then executing processing of the second series by manual execution, for example. In this case, the update function 146 determines that the number of series is 2 because the first series includes the feature α, or updates the examination end determination rule ERL to the timeout time obtained by summing the timeout times of the first series and the second series.

Alternatively, it is assumed that an examination includes three series, the first series includes a feature γ, the second and third series do not include the feature γ, and in automatic execution, processing of the first series is executed, followed by processing of the second and third series. In this case, the update function 146 updates the examination end determination rule ERL assuming that the number of series of the examination is 3 when the first series includes the feature γ.

In this manner, the medical information processing device 100 ends the processing shown in FIG. 6. In the medical information processing device 100 of the above embodiment, the examination end determination rule ERL is updated on the basis of results of processing by the past execution application. This makes it possible to reduce waste in processing in the application. In addition, since it is possible to avoid setting an unnecessary timeout time, it is possible to prevent situations in which the time required for processing is wasted overall and to reduce the impact on performance.

In the above embodiment, when processing DICOM images of a plurality of series in an examination, the processing order is set among the plurality of series, but for example, when processing DICOM images of a plurality of examinations, the order may be set among the plurality of examinations. The order may also be set among a plurality of series in an examination and among a plurality of examinations.

According to at least one of the embodiments described above, the medical image processing device has a selection function that automatically selects an execution application to be executed on a medical image that is a processing target from among a plurality of applications, an execution unit that executes the execution application, a setting unit that sets execution information regarding execution of processing of the processing target by the execution application on the basis of a result of referring to the examination end determination rule for supplementary information attached to the medical image, and an update unit that updates the examination end determination rule on the basis of a past result of processing by the execution application, thereby reducing wasteful processing in applications.

The setting function 144 of the medical information processing device 100 describe above may be included in the terminal device 10 which displays various information provided by the medical information processing device 100. For example, a dedicated application program (viewer) installed in the terminal device 10 executes the setting function 144, refers to the examination end determination rule ERL for a DICOM tag attached to the DICOM image output from the modality 20, and sets execution information (for example, the number of examination series, the timeout time, the processing start timing, and the processing end timing). The information of the DICOM tag and the examination end determination rule ERL may be provided from the medical information processing device 100. A display control function of the terminal device 10 displays the execution information set by the setting function 144 on the viewer (display). A user such as a doctor checks the page (screen) including the execution information displayed on the terminal device 10, and can understand the content of the analyzing process by the medical information processing device 100 (for example, the timing when all of the analyzing results completely appear). In such a configuration, the terminal device 10 is an example of a medical information processing device for displaying a result of processing of a medical image by an application.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical information processing device, comprising processing circuitry configured to:
automatically select, from among a plurality of applications, an execution application to be executed on a medical image that is a processing target;
execute the execution application;
set execution information regarding execution of processing of the processing target by the execution application on the basis of a result of referring to an examination end determination rule for supplementary information attached to the medical image; and
update the examination end determination rule on the basis of a past result of processing by the execution application.

2. The medical information processing device according to claim 1, wherein the processing circuitry is configured to update the examination end determination rule on the basis of a result of processing by the execution application manually operated by a user

3. The medical information processing device according to claim 1, wherein the processing circuitry is configured to update the examination end determination rule on the basis of a result of processing by the execution application after a certain period of time has elapsed since processing by the execution application has ended.

4. The medical information processing device according to claim 1, wherein the examination end determination rule includes a timeout time at which an acceptance time for accepting medical images to be processed collectively by the execution application ends, and
the processing circuitry is configured to set execution information for starting processing of the processing target by the execution application when the timeout time has expired.

5. The medical information processing device according to claim 4, wherein the processing circuitry is configured to set at least one of a timing for starting processing by the execution application, a timing for ending processing by the execution application, or the timeout time as the execution information.

6. The medical information processing device according to claim 1, wherein the processing circuitry is configured to set an execution order for executing a plurality of medical images when the plurality of medical images are executed by the execution application.

7. The medical information processing device according to claim 6, wherein the supplementary information includes a priority regarding the order in which the medical images are executed.

8. The medical information processing device according to claim 7, wherein the processing circuitry is configured to:
estimate a processing time required for processing the medical images by the execution application; and
set an execution order for executing the plurality of medical images on the basis of the priority and the processing time.

9. The medical information processing device according to claim 8, wherein the processing target is processable by the execution application and includes a first medical image acquired earlier and a second medical image acquired later, and
when processing of the second medical image by the execution application ends before processing of the first medical image can be started, the processing circuitry is configured to set a timing of processing of the second medical image by the execution application to be before processing of the first medical image by the execution application regardless of the priority.

10. The medical information processing device according to claim 1, wherein
the supplementary information includes a DICOM tag, and
the processing circuitry is configured to set the execution information on the basis of similarity between a DICOM tag attached to the medical image and a DICOM tag defined in the examination end determination rule.

11. The medical information processing device according to claim 10, wherein the processing circuitry is configured to:
weight each of a plurality of DICOM tags; and
calculate the similarity on the basis of the weighted plurality of DICOM tags.

12. A medical information processing device for displaying a result of processing of a medical image by an application, comprising processing circuitry configured to:
set execution information regarding execution of the processing of the medical image by the application on the basis of a result of referring to an examination end determination rule for supplementary information attached to the medical image; and
display the set execution information on a display.

13. A medical information processing method, using a computer, comprising:
automatically selecting and executing, from among a plurality of applications, an execution application to be executed on a medical image that is a processing target;
setting execution information regarding execution of processing of the processing target by the execution application on the basis of a result of referring to an examination end determination rule for supplementary information attached to the medical image; and
updating the examination end determination rule on the basis of a past result of processing by the execution application.

14. A medical information processing method, using a computer of a medical information processing device for displaying a result of processing of a medical image by an application, the medical information processing method comprising:
setting execution information regarding execution of the processing of the medical image by the application on the basis of a result of referring to an examination end determination rule for supplementary information attached to the medical image; and
displaying the set execution information on a display.

15. A computer-readable non-transitory recording medium storing a program causing a computer to:
automatically select and execute, from among a plurality of applications, an execution application to be executed on a medical image that is a processing target;
set execution information regarding execution of processing of the processing target by the execution application on the basis of a result of referring to an examination end determination rule for supplementary information attached to the medical image; and
update the examination end determination rule on the basis of a past result of processing by the execution application.

16. A computer-readable non-transitory recording medium storing a program causing a computer of a medical information processing device for displaying a result of processing of a medical image by an application to:
set execution information regarding execution of the processing of the medical image by the application on the basis of a result of referring to an examination end determination rule for supplementary information attached to the medical image; and
display the set execution information on a display.
